(19) United States
(12) Patent Application Publication
(10) Pub. No.: US 2010/0227388 A1
(43) Pub. Date: Sep. 9, 2010

(54) Reactor for growing cultures

(76) Inventors: Oleg Shvabsky, Hallandale Beach, FL (US); Jacob Gitman, Bay Harbor Island, FL (US)

Correspondence Address: l. Zborovsky, 16 Bantry Ct, Huntington, NY, 11743, US

(21) Appl. No.: 12/380,805

(22) Filed: Mar. 4, 2009

(51) Int. Cl. C12M 1/00

(52) U.S. Cl. 435/292.1

(57) ABSTRACT

A reactor for growing a culture has a plurality of sections located above each other in a vertical direction and each having a peripheral surface, a bottom, and a top forming together a container for accommodating a solution for growing a culture, and a plurality of light sources arranged on the top so that they emit light into an interior of the container.

### BACKGROUND OF THE INVENTION

The present invention relates to reactors for growing agro-cultures.

Devices or reactors for intense growing of cultures of this type include devices of two types.

The first type of the devices are devices which are tank structures for accommodating a solution of a culture to be grown and is provided with light sources that are isolated in glass pipes. These devices have several disadvantages. It is characterized by illumination losses due serial arrangement of light sources which screen one another. The lower light sources are used inefficiently, since during the process of growth of the cultures the culture moves down and the solution which becomes darker prevents passage of light. It is difficult to control the light sources which are no longer operational and to change them. It is necessary to withdraw heat from light sources, in order to maintain optimal temperature for growth of the cultures. There are problems for regulation of intensity and spectrum of a light flow due to restrictive location of the pipes inside the tank. Finally, it is necessary to periodically clean the surfaces of the glass pipes from products of biological synthesis that are sticking to them.

The second types of the devices are devices which have a tubular structure, in which a solution with culture is located inside the tube, while the light sources are located outside. They also have certain disadvantages. In particular, a gap between the pipes and dispersion due to the location of the sources in an open space constitute a part of the light flow which is not used. The pipes have to be illuminated from both sides. A geometrical coefficient of light loss from each side of a cylindrical pipe is approximately 1.57. Finally, the cylindrical surface of the glass pipe reflects a significant part of the supplied light.

### SUMMARY OF THE INVENTION

It is therefore an object of the present invention to provide a reactor for growing cultures, which avoids the disadvantages of the prior art.

In keeping with these objects and with others which will become apparent hereinafter, one feature of the present invention resides, briefly stated, in a reactor for growing a culture, comprising a plurality of sections located above each other in a vertical direction and each having a peripheral surface, a bottom, and a top forming together a container for accommodating a solution for growing a culture; and a plurality of light sources arranged on said top so that they emit light into an interior of the container.

Another feature of the present invention resides, briefly stated, in said light sources are light sources selected from the group consisting of sunlight sources, electrical light sources and both.

A further feature of the present invention resides, briefly stated, in said bottom has an inner surface facing toward the interior of each container and provided with a light-reflective coating.

Still a further feature of the present invention resides, briefly stated in cleaning means for cleaning an inner surface of each of said containers.

An additional feature of the present invention resides, briefly stated, in a common substantially vertical shaft extending over a height of all said sections and rotatably connected with a central axial provided in each of said sections.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is an elevational view of a biological reactor in accordance with the present invention;

FIG. 2 is a view showing a cross-section of one section of the biological reactor in accordance with the present invention;

FIG. 3 is a view showing another cross-section of the section of the biological reactor in a direction which is perpendicular to the direction of the cross-section of FIG. 2;

FIGS. 4,5,6, are two views of a central part of the section of the inventive biological reactor, on an enlarged scale.

FIG. 7 is a view showing an internal surface of a biological reactor as seen from above;

FIG. 8 is a view showing a surface of a lid of the inventive biological reactor with light sources arranged on it; and

FIG. 9 is a plan view of section which is turned away from the biological reactor for servicing;

FIG. 10 shows the grate.

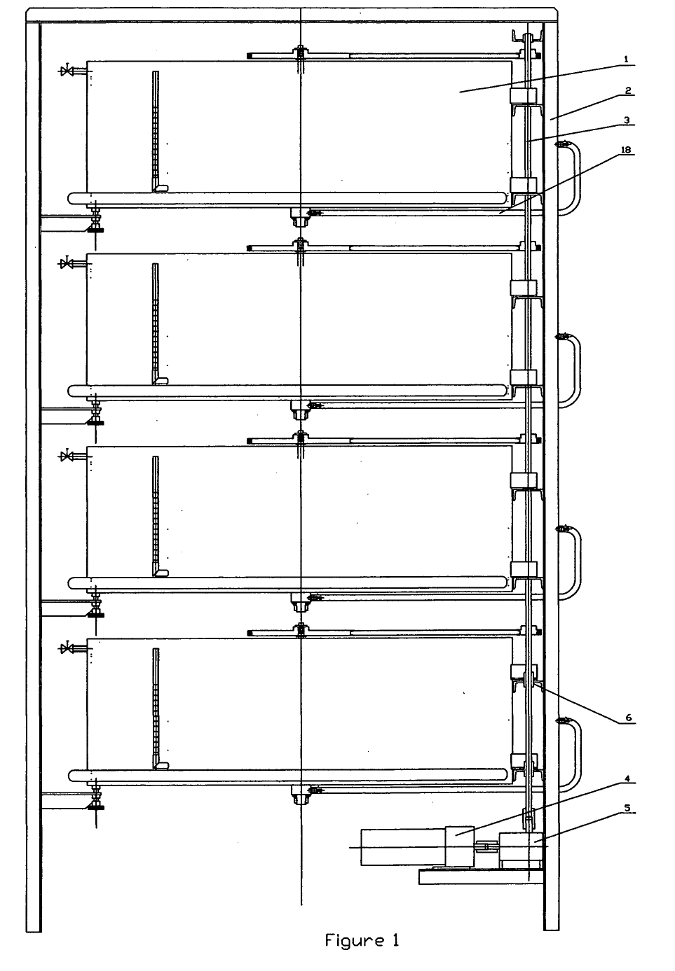

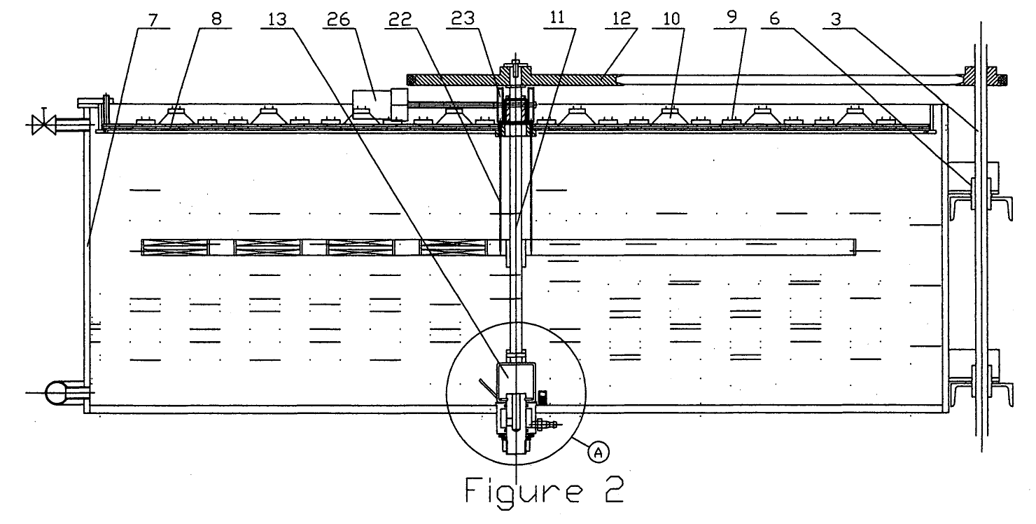

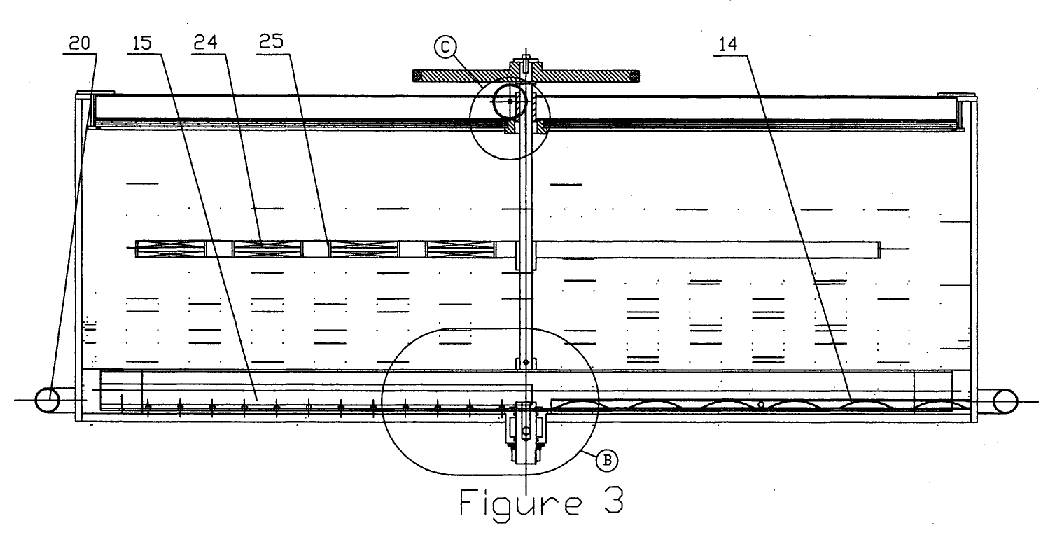

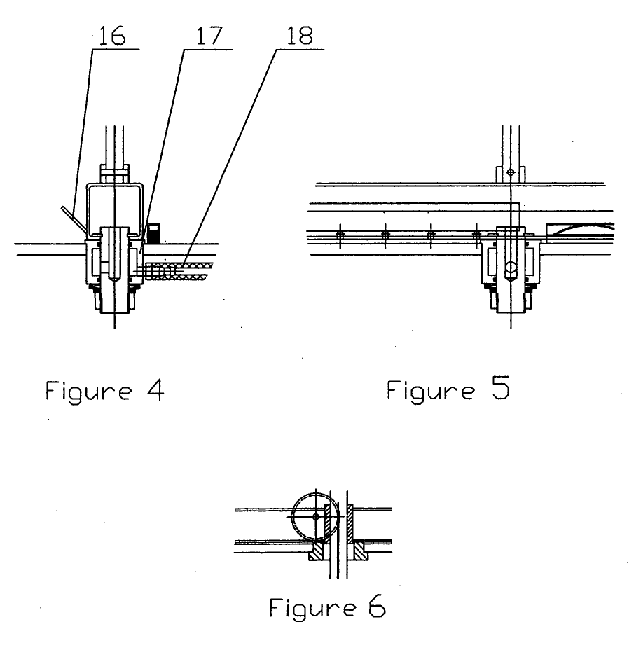

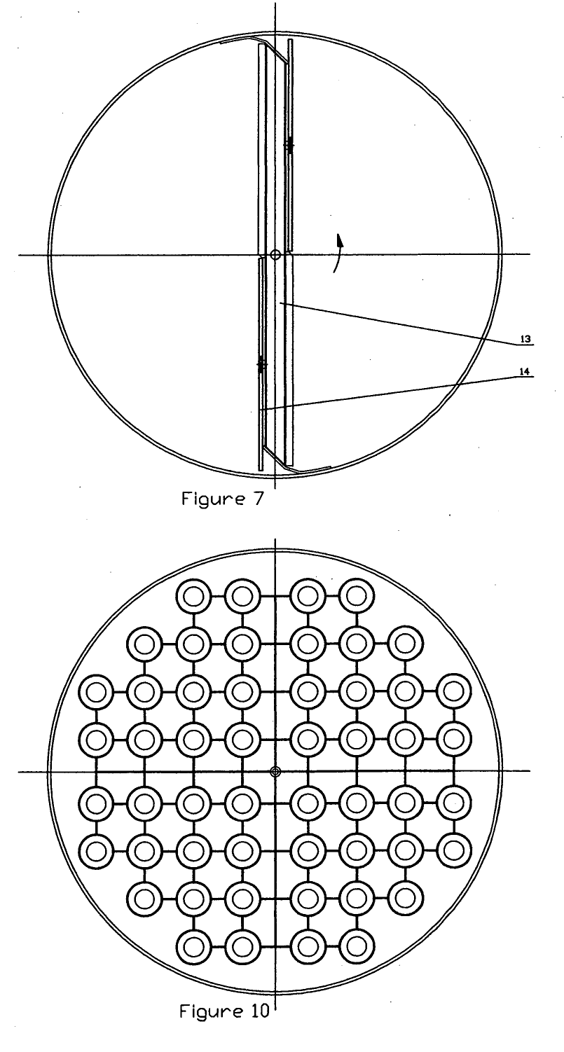

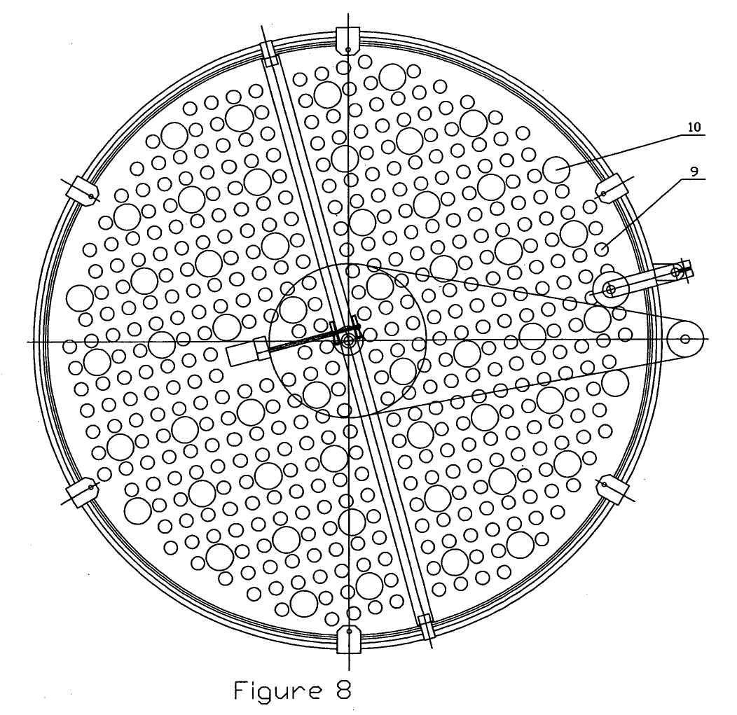

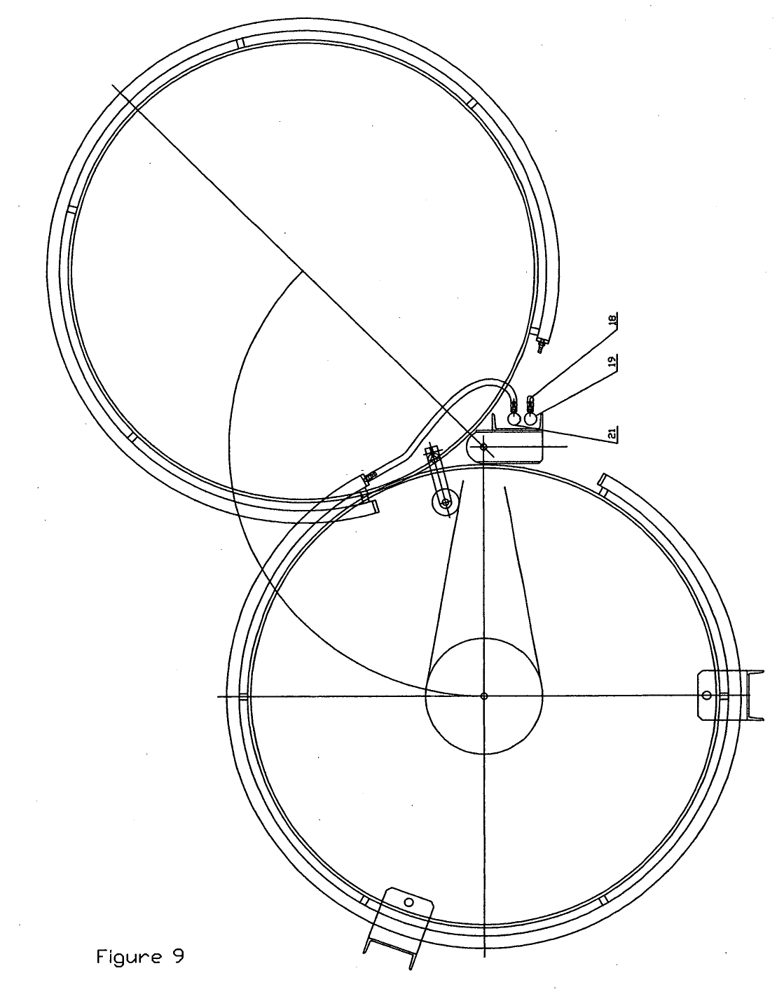

### DESCRIPTION OF THE PREFERRED EMBODIMENTS

A biological reactor for growing cultures, for example agricultural cultures are formed as a vertical tank, including a plurality of sections identified with reference numeral 1. The sections are located one above the other, and their number is limited only by a height of a space in which the biological reactor is accommodated.

The biological reactor further has a frame 2 and surrounding the sections 1. A substantially vertically shaft 3 is connected to the frame 2 and rotates by an electric motor 4 through a reducing transmission 5. A plurality of turning bushings 6 are provided and turnably connected with the shaft 3.

Each section 1 has a container 7 which is closed from above by a lid, preferably a glass lid 8. A plurality of light sources formed as light emitting elements are provided in the lid or on the lid so that emit light toward the interior of the lid container 7. The light sources can be formed as a light diode 9, as a sun light sources 10, and both.

It is to be understood that other light sources can be provided as well.

It is to be understood that light sources, can be connected with a power supply.

An axle 11 is located in a center of each container 7. It is connected with the shaft 3 by a belt transmission 12. While the light sources are located from the lid 8, a bottom 22 of each tank 7 is provided with a light reflecting coating 23.

Devices 13 for collecting a product are arranged on the axle 11. They are provided with guiding elements 16 for guiding a flow of the product toward each device. Cleaning devices 14 and 15 are provided for cleaning of the container 7 and composed of cleaning elements for example similar to windshield. wipers and the like.

The product collecting device 13 is connected, via a collector 17, by means of a hose 18 with a pumping out pipe 19 connected to a corresponding pump.

A collector 20 is arranged around each container 7, with which, through a pipe 21, a fresh solution is introduced.

The reactor operates in the following manner; each section 1 of the reactor is loaded with a solution of a necessary concentration for growing a biological mass in it. During growing of the biological mass an illumination is provided by light sources 9, and/or 10 emitting light into the interior of each section 1. The axle 11 of which section 1 rotates from the shaft 3 via the belt transmission 12. The product is collected by the device for collecting the product 13 supplied with the aid of the guides 16. The container 7 of each section 1 is cleaned by the elements 14 and 15. The product produced in the process of photosynthesis sinks to the bottom and continuously or with a pulse frequency is removed for subsequent operations from the devices 13 through the collector 17 via the hose 18 connected with a pipe for pumping out of the product.

The light sources located above the surface of the solution can be used in a mode of sunlight by means of fiber-optics light guides, electrical sources such as light diodes, lasers, mazers, or in any combination of the above.

Spectrum, frequency and intensity of the light sources can be easily adjusted depending on the culture to be grown in the biological reactor.

These features eliminate the above mentioned disadvantages of the prior art.

Each section can be turned outwardly from the interior of the container outwardly through the bush 6 for service.

The inner surfaces of the containers of the sections are cleaned from products of photosynthesis.

All processes in the reactor are performed in a continuous cycle.

It will be understood that each of the elements described above, or two or more together, may also find a useful application in other types of constructions differing from the type described above.

While the invention has been illustrated and described as embodied in a reactor for growing cultures, it is not intended to be limited to the details shown, since various modifications and structural changes may be made without departing in any way from the spirit of the present invention.

Without further analysis, the foregoing will so fully reveal the gist of the present invention that others can, be applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. A reactor for growing a culture, comprising a plurality of sections located above each other in a vertical direction and each having a peripheral surface, a bottom, and a top forming together a container for accommodating a solution for growing a culture; and a plurality of light sources arranged on said top so that they emit light into an interior of the container.

2. A reactor as defined in claim 1, wherein said light sources are light sources selected from the group consisting of sunlight sources, electrical light sources and both.

3. A reactor as defined in claim 1, wherein said bottom has an inner surface facing toward the interior of each container and provided with a light-reflective coating.

4. A reactor as defined in claim 1, further comprising cleaning means for cleaning an inner surface of each of said containers.

5. A reactor as defined in claim 1, further comprising a common substantially vertical shaft extending over a height of all said sections and rotatably connected with a central axial provided in each of said sections.

6. A reactor as defined in claim 5, further comprising bushing means connecting each of said sections with said shaft, so that each of said containers can be turned from the interior of said reactor outwardly for service purposes.

7. A reactor as defined in claim 1, further comprising means for collecting a product provided with a guide for guiding a flow of the product into said collecting means.

8. A reactor as defined in claim 7, further comprising means for connecting each of said connectors with a pipe for pumping out the product from each of said containers.

9. A reactor as defined in claim 1, further comprising a collector arranged around each of said containers for supplying a fresh solution into each of said containers.
